SEQUENCE LISTING (6 sequences)

SEQ ID NO: 1 - protein, 348 aa, Saccharomyces cerevisiae
MTTEKTVVFVSGATGFIALHVVDDLLKTGYKVIGSGRSQEKNDGLLKKFKSNPNLSMEIVEDIAAPNAFDKVFQKHGKEIKVVLHIASPVHFNTTDFEKDLLIPAVNGTKSILEAIKNYAADTVEKVVITSSVAALASPGDMKDTSFVVNEESWNKDTWESCQANAVSAYCGSKKFAEKTAWDFLEENQSSIKFTLSTINPGFVFGPQLFADSLRNGINSSSAIIANLVSYKLGDNFYNYSGPFIDVRDVSKAHLLAFEKPECAGQRLFLCEDMFCSQEALDILNEEFPQLKGKIATGEPGSGSTFLTKNCCKCDNRKTKNLLGFQFNKFRDCIVDTASQLLEVQSKS

SEQ ID NO: 2 - DNA, 1047 bp, Saccharomyces cerevisiae
atgactactgaaaaaaccgttgtttttgtttctggtgctactggtttcattgctctacacgtagtggacgatttattaaaaactggttacaaggtcatcggttcgggtaggtcccaagaaaagaatgatggattgctgaaaaaatttaagagcaatcccaacctttcaatggagattgtcgaagacattgctgctccaaacgcttttgacaaagtttttcaaaagcacggcaaagagatcaaggttgtcttgcacatagcttctccggttcacttcaacaccactgatttcgaaaaggatctgctaattcctgctgtgaatggtaccaagtccattctagaagcaatcaaaaattatgccgcagacacagtcgaaaaagtcgttattacttcttctgttgctgcccttgcatctcccggagatatgaaggacactagtttcgttgtcaatgaggaaagttggaacaaagatacttgggaaagttgtcaagctaacgcggtttccgcatactgtggttccaagaaatttgctgaaaaaactgcttgggattttctcgaggaaaaccaatcaagcatcaaatttacgctatcaaccatcaacccaggatttgtttttggccctcagctatttgccgactctcttagaaatggaataaatagctcttcagccattattgccaatttggttagttataaattaggcgacaatttttataattacagtggtccttttattgacgttcgcgatgtttcaaaagctcatttacttgcatttgagaaacccgaatgcgctggccaaagactattcttatgtgaagatatgttttgctctcaagaagcgctggatatcttgaatgaggaatttccacagttaaaaggcaagatagcaactggcgaacctggtagcggctcaacctttttgacaaaaaactgctgcaagtgcgacaaccgcaaaaccaaaaatttattaggattccaatttaataagttcagagattgcattgtcgatactgcctcgcaattactagaagttcaaagtaaaagctaa

SEQ ID NO: 3 - protein, 347 aa, Saccharomyces cerevisiae
MTTDTTVFVSGATGFIALHIMNDLLKAGYTVIGSGRSQEKNDGLLKKFNNNPKLSMEIVEDIAAPNAFDEVFKKHGKEIKIVLHTASPFHFETTNFEKDLLTPAVNGTKSILEAIKKYAADTVEKVIVTSSTAALVTPTDMNKGDLVITEESWNKDTWDSCQANAVAAYCGSKKFAEKTAWEFLKENKSSVKFTLSTINPGFVFGPQMFADSLKHGINTSSGIVSELIHSKVGGEFYNYCGPFIDVRDVSKAHLVAIEKPECTGQRLVLSEGLFCCQEIVDILNEEFPQLKGKIATGEPATGPSFLEKNSCKFDNSKTKKLLGFQFYNLKDCIVDTAAQMLEVQNEA

SEQ ID NO: 4 - DNA, 1044 bp, Saccharomyces cerevisiae
atgactactgataccactgttttcgtttctggcgcaaccggtttcattgctctacacattatgaacgatctgttgaaagctggctatacagtcatcggctcaggtagatctcaagaaaaaaatgatggcttgctcaaaaaatttaataacaatcccaaactatcgatggaaattgtggaagatattgctgctccaaacgcctttgatgaagttttcaaaaaacatggtaaggaaattaagattgtgctacacactgcctccccattccattttgaaactaccaattttgaaaaggatttactaacccctgcagtgaacggtacaaaatctatcttggaagcgattaaaaaatatgctgcagacactgttgaaaaagttattgttacttcgtctactgctgctctggtgacacctacagacatgaacaaaggagatttggtgatcacggaggagagttggaataaggatacatgggacagttgtcaagccaacgccgttgccgcatattgtggctcgaaaaagtttgctgaaaaaactgcttgggaatttcttaaagaaaacaagtctagtgtcaaattcacactatccactatcaatccgggattcgtttttggtcctcaaatgtttgcagattcgctaaaacatggcataaatacctcctcagggatcgtatctgagttaattcattccaaggtaggtggagaattttataattactgtggcccatttattgacgtgcgtgacgtttctaaagcccacctagttgcaattgaaaaaccagaatgtaccggccaaagattagtattgagtgaaggtttattctgctgtcaagaaatcgttgacatcttgaacgaggaattccctcaattaaagggcaagatagctacaggtgaacctgcgaccggtccaagctttttagaaaaaaactcttgcaagtttgacaattctaagacaaaaaaactactgggattccagttttacaatttaaaggattgcatagttgacaccgcggcgcaaatgttagaagttcaaaatgaagcctaa

SEQ ID NO: 5 - DNA, 80 bp, Artificial Sequence
  Description of Artificial Sequence Synthetic primer
atgactactgataccactgttttcgtttctggcgcaaccggtttcattgctctacacattcctttttcaatgggtaataa

SEQ ID NO: 6 - DNA, 80 bp, Artificial Sequence
  Description of Artificial Sequence Synthetic primer
ttaggcttcattttgaacttctaacatttgcgccgcggtgtcaactatgcaatcctttaatagcttttcaattcaattca

(19) United States
(12) Patent Application Publication
(10) Pub. No.: US 2006/0005282 A1
(43) Pub. Date: Jan. 5, 2006

(54) Production and use of salt tolerant and culture density tolerant organisms

(75) Inventor: Jonathan Rosenblum, San Diego, CA (US)

Correspondence Address: FOLEY & LARDNER LLP, P.O. BOX 80278, SAN DIEGO, CA, 92138-0278, US

(73) Assignee: Activx Biosciences, Inc

(21) Appl. No.: 10/512,277

(22) PCT Filed: Jan. 16, 2003
(86) PCT No.: PCT/US03/01286

§ 371 Date: May 23, 2005

Related U.S. Application Data
(60) Provisional application No. 60/375,083, filed on Apr. 23, 2002.

(51) Int. Cl. A01H 1/00; C12N 1/16; C12N 1/18; C12N 5/04

(52) U.S. Cl. 800/289; 435/254.2; 435/254.3; 435/419

(57) ABSTRACT

Cells and organisms tolerant to high salt and/or high culture density are described. Such cells have two particular genes inactivated, resulting in the environmental stress tolerance. Also described are methods for creating and using such cells and organisms. The cells and organisms can be used in many different applications where the tolerance of high salt and/or high density is advantageous. Examples include industrial and food product processes involving cell culture, e.g., yeast culture.

### BACKGROUND OF THE INVENTION

The present invention relates generally to the field of organisms that tolerate environmental stress conditions, in particular high salt and/or high culture density conditions.

The following discussion of the background of the invention is provided merely to aid the reader in understanding the disclosure, and is not admitted to describe or constitute prior art to the present invention.

The presence or accumulation of various salts, e.g., sodium chloride, or other dissolved components can seriously inhibit growth of organisms exposed to such conditions. In plants, for example, high salt can interfere with water absorption, inhibit the absorption of other necessary ions, and cause loss of turgor pressure and stoma closure, inhibiting photosynthesis and growth. Likewise, in organisms such as yeast, high salt medium can cause dehydration of the cells, as well as interfere with metabolism, again causing growth inhibition. The provision of salt-tolerant organisms is therefore useful in allowing growth of the organisms under adverse conditions that normally would not support a useful level of growth, or not support growth at all. A number of different techniques have been used to provide such salt tolerant organisms.

In an approach not involving direct manipulation of DNA, plants have been selected for salt tolerance using conventional selection methods. See, e.g., Dobrenz et al., U.S. Pat. No. 6,005,165, entitled “Salt Tolerant Alfalfa”. In addition, salt tolerance in plant cells and callus has been selected in vitro. For example, see Dix, The Plant Journal 3:309-313 (1993).

In addition, certain genes associated with salt tolerance have been identified. Expression of these genes increases the salt tolerance in the organism. See, e.g., Gaxiola et al., EMBO J. 11:3157-3164 (1992); Glaser et al, EMBO J. 12:3105-3110 (1993); and Muriaga et al., Science 259:508-510 (1995). In some cases, such expression is heterologous expression, e.g., in yeast. An example of heterologous expression is provided in Gasser et al., U.S. Pat. No. 5,859,337, entitled “Genes Conferring Salt Tolerance and Their Uses”, which describes expression of certain Arabidopsis genes in yeast.

Similarly, Murata, U.S. Pat. No. 6,281,412, entitled “Method for Creating Osmotic-Pressure-Tolerant Plant” describes the heterologous expression of a choline oxidase gene to provide salt tolerant plants. In that case, expression of the choline oxidase gene catalyzes the conversion of choline to glycinebetaine, which acts as a compatible solute, thereby enhancing salt tolerance.

The expression (or overexpression) of antiporter genes can also provide salt tolerance. For example, Waditee et al. Proc. Natl. Acad. Sci. USA 99:4109-4114 (2002) describes expression of a Na+/H+ antiporter gene from a halotolerant cyanobacterium in a freshwater cyanobacterium, resulting in enhanced salt tolerance. Similarly, creation of salt tolerance in plants by expression of sodium antiporters has been demonstrated. For example, Zhang et al., Proc. Natl. Acad. Sci. USA 98:12832-12836 (2001) described sodium antiporter expression and salt tolerance in Brassica napus and Zhang et al., Nat. Biotechnol. 19:765-768 (2001) described similar effects in tomato. Likewise, Apse et al., Science 285:1256-1258 (1999) described sodium antiporter expression and salt tolerance in Arabidopsis.

Another approach involves the application of a substance that induces salt tolerance. An example of such a substance is gibberellin. Zhao Ke-fu et al., Aust. J. Plant Physiol., 13:547-551 (1986). Another such substance is 5-aminolevulinic acid (or its salts). Application of these compounds is indicated to increase salt tolerance. Kuramochi, et al., U.S. Pat. No. 5,661,111, entitled “Method for Improving Plant Salt Tolerance”.

Yeast can also be obtained that are salt tolerant. For example, Park et al., J. Biol. Chem. 276:28694-28699 (2001) indicates that continuous activation of calcineurin in yeast provides a salt tolerant phenotype by inducing increased expression of the PMR2 gene.

### SUMMARY OF THE INVENTION

The present invention provides methods for producing and using cells that tolerate environmental stress conditions, particularly high salt and/or high culture density conditions, as well as the cells themselves, and related compositions. Such cells are generally provided by inactivating particular genes. This allows modified strains to be created from many different cells and organisms, for example, yeasts and plants. The resulting modified cells and organisms can be used in a large variety of processes in which tolerance to high salt, high culture density, and/or other environmental stress conditions is advantageous.

Thus, in a first aspect, the invention provides a cell(s), preferably an isolated or purified cell, that has at least one gene functionally homologous to YGLO39w, or functionally homologous to YGL157w or both inactivated. As a result of the inactivation of the gene, the cell has greater tolerance to high salt, e.g., sodium chloride (and preferably additional dissolved materials and/or high culture density), concentration than a corresponding cell that has an active form of the gene. In some embodiments, a plurality of genes are inactivated, e.g., 2, 3, 4, or even more genes.

Such gene activation and resulting salt tolerance can be obtained in a variety of different cells. Thus, in various embodiments, the cell is a yeast cell, preferably a Saccharomyces cell, most preferably a Saccharomyces cerevisiae cell. Likewise, the cell may be from an Aspergillus species. More generally the cell can be a fungal cell or a plant cell. In preferred embodiments, the cell is in a cell culture, preferably in a population of such cells. Preferably the cell culture is a liquid culture. In preferred embodiments, the cell culture is a high density cell culture.

In preferred embodiments, the cell is in high salt culture conditions; the cell(s) further comprises at least one additional genetic characteristic providing increased salt tolerance, e.g., the additional genetic characteristic can be a mutation(s) or a heterologous gene(s) that may, for example, provide increased sodium pump (antiporter) activity; in addition to increased salt tolerance, the cell is also tolerant to increased culture density and/or starvation conditions as compared to corresponding cells that have active forms of the genes functionally homologous to YGLO39w and/or YGL157w, preferably tolerating increased culture densities where the density is increased at least 25%, 50%, 100%, 150%, 200%, 300%, 400%, 500%, 700%, 1000%, or even more; and/or the cells maintain active growth to a higher density than cells with active forms of the genes, e.g., the cells can maintain exponential growth phase to a higher density. In certain embodiments, the cell (or culture or population) is in a culture that is both high salt and high density. The embodiments indicated above also apply to the other aspects of the present invention unless indicated to the contrary, e.g., by the aspect specifying a limited class of cell types.

In a related aspect, the invention provides a population of salt tolerant cells in a high salt environment, where the cells have at least one inactivated gene functionally homologous to YGLO39w or YGL157w or both, thereby providing salt tolerance to the cells. The salt tolerant cells will therefore grow in the high salt environment.

In another related aspect, the invention provides a salt tolerant organism that has cells in which at least one gene functionally homologous to YGL039w or YGL157w or both are inactivated. Highly preferably, the inactivation of the genes confers salt tolerance and/or an ability to grow to high density and/or tolerance to solutions with elevated dissolved components (e.g., various ions (such as various minerals) and/or products of cell or organism culture).

In another aspect, the invention provides yeast that have at least one artificial genetic alteration, where the yeast is more salt tolerant and actively grows to higher culture density than an isogeneic yeast without the at least one artificial genetic alteration. Such alterations include those described above, e.g., an alteration inactivating at least one gene.

In a related aspect, the invention provides a yeast strain that is more salt tolerant and grows to a higher density in rich media than the corresponding wild type strain. Preferably the corresponding wild type strain is a designated standard wild type strain deposited in an approved depository.

In another related aspect, the invention includes a Saccharomyces cerevisiae yeast strain that grows to an OD600 of at least 15 in YPD medium. Preferably the strain actively grows to higher density in YPD medium with 0.9M NaCl than strain BY4743

Yet another related aspect concerns a Saccharomyces cerevisiae yeast strain that grows to a higher density and/or faster in YPD medium with 0.9M NaCl than strain BY4743.

Another related aspect concerns a mutated cell that is more tolerant to the presence of salt and grows to higher density under growth conditions than the parent cell. The mutation can include inactivation of at least one gene, e.g., a gene functionally homologous to YGLO39w and/or YGL157w.

In another aspect, the invention provides a culture kit that includes a plurality of cells as described for an aspect above, e.g., cells comprising at least one inactivated gene functionally homologous to YGLO39w and/or YGL157w, packaged in a storage device. Preferably the kit also includes instructions for growing the cells and/or one or more culture media suitable for growing the cells. A medium may, for example, be provided in liquid form (concentrated or at normal use concentration), in dry form, or in the form of a plurality of components that are combined to at least partially provide a medium. A medium may also be a high salt medium.

In preferred embodiments, the cells are of a type as described for an aspect above, or otherwise described herein as suitable for use in the present invention. In kits containing fungal cells, the fungal cells may be spores. Likewise, in kits containing plant cells, the plant cells may be seeds, seedlings, cuttings, or other plant form capable of growth. The storage device may, for example, include a box(es), vial(s), tube(s), envelope(s), can(s), and/or bag(s).

In another aspect, the invention provides a method for growing cells to high density, by culturing cells that have at least one inactivated gene functionally homologous to genes YGLO39w and/or YGL157w under growth conditions, until high cell density is obtained during exponential growth phase. That high cell density is greater than that obtained in exponential growth phase under the same growth conditions for cells that are isogeneic except for having active genes functionally homologous to genes YGLO39w and YGL157w, preferably at least 25%, 50%, 80%, 100%, 200% greater, or even higher. In preferred embodiments, the cells are of a type described for an aspect above, or otherwise described herein as suitable for use in the present invention.

In certain embodiments, the cells are grown to stationary phase. Preferably the cell density obtained at stationary phase is greater than that obtained at stationary phase under the same growth conditions for cells that are isogeneic except for having active genes functionally homologous to genes YGLO39w and YGL157w, e.g., at least 25%, 50%, 80%, 100%, or 200% higher, or even greater.

In another aspect, the ability of cells of the present invention to grow to higher densities (during active growth and/or final cell density) provides a method for providing increased yield of a cell product. The method involves culturing cells that have at least one inactivated gene functionally homologous to genes YGLO39w and/or YGL157w under growth conditions, and purifying the desired product or products. In preferred embodiments, the cells are grown in exponential growth phase to a cell density at least 25%, 50%, 80%, 100%, or 200% greater (or even more) than that obtained in exponential growth phase under the same growth conditions for cells that are isogeneic except for having active genes functionally homologous to genes YGLO39w and/or YGL157w; the cells are grown to stationary phase, preferably the density of said cells at stationary phase is greater than that obtained at stationary phase under the same growth conditions for cells that are isogeneic except for having active genes homologous to genes YGLO39w and YGL157w, e.g., at least 25%, 50%, 80%, 100%, or 200% greater. Preferably the cells are of a types indicated for an aspect above, or otherwise indicated herein as suitable for use in the present invention.

Likewise, in a related aspect, the invention provides a method for growing cells in high salt conditions, by culturing cells that have at least one inactivated gene functionally homologous to genes YGLO39w and/or YGL157w under high salt growth conditions, where the high salt growth conditions inhibit growth of cells that are isogeneic except for having active genes functionally homologous to genes YGLO39w and/or YGL157w. The level or concentration of ions, e.g., sodium ions, in the high salt conditions can vary depending on the particular type of cells. In particular embodiments, the sodium concentration (e.g., from sodium chloride) is 0.9 M (molar)±0.1 M, or at least 0.9 M. In preferred embodiments, cells are of a type indicated herein as suitable for the present invention.

In another aspect, the invention provides a method for producing a comestible product in a process utilizing cell culture, involving culturing cells that have at least one inactivated gene functionally homologous to YGLO39w and/or YGL157w in that process. Preferably the cells are as described for an aspect above. Examples of comestible products that utilize a cell culture process include soy sauce (fermented), beer, wine, cheese, and certain nutritional supplements, e.g., yeast-based nutritional supplements. The cells used in the process can be of various types, selected as suitable for the particular process, including cells of types described herein as suitable for use in the present invention.

In some cases, the process is a fermentation. Typically the culturing is carried out in liquid culture. The culturing can be performed as continuous active culture, or carried out in batch culture, e.g., to stationary phase. In preferred embodiments, the cell density in a batch culture, e.g., at stationary phase, or in the continuous active culture is greater than that obtained under the same growth conditions by cells that are isogeneic except for having active genes functionally homologous to genes YGLO39w and/or YGL157w.

Two related aspects provide a method for producing a cell tolerant to high levels of dissolved materials, and a method for producing a cell tolerant to high culture density. The methods involve inactivating forms of genes having sequence match to YGLO39w and/or YGL157w in the cell; and testing those cells for tolerance to high levels of dissolved material, e.g., salts such as sodium chloride, and/or high culture density. The type of cells can be of many different types, e.g., as indicated herein for use in the present invention.

As recognized by persons familiar with molecular biological techniques, the gene inactivations can be accomplished in various ways. Examples include, without limitation, deletion of at least a part of the genes; creating insertions in the genes, typically heterologous sequence insertions; inhibiting transcription; inhibiting transcript processing; and inhibiting translation, as well as inhibition of the polypeptide or protein. For use in production of comestible products and other applications in which long term stability of the inactivation is advantageous, preferably deletion or insertion inactivations are used.

Different types of cells can have a plurality of genes, e.g., 2 or more than 2 genes with sequence match to YGLO39w and/or YGL157w, for example, 3, 4, 5, or more genes (and/or gene products) with matching sequences. For example, S. cerevisiae contains 4 different genes having matching sequences, but YGLO39w and YGL157w are the genes found to provide tolerance to salt and high culture density when inactivated. Thus, in certain embodiments, the cell contains at least 2, 3, 4, or 5 different genes with a sequence match to YGLO39w and/or YGL157w. Cells with different combinations of those genes inactivated, e.g., pairwise combinations) are tested to identify the functional homologs. Preferably cells with at least all pairwise combinations of inactivated genes are tested, and more preferably with all combinations of inactivated genes are tested.

The high levels of dissolved materials can include a variety of different components, e.g., high levels of sodium chloride and/or high levels of cell culture products.

The method can also include identifying genes and/or gene products with sequence match to YGLO39w or YGL157w or both (or the corresponding gene products). Such identifications can be done in a number of different ways. Appropriate methods are well-known to those in the art. Examples include performing sequence alignments, using low stringency PCR to amplify sequences with sequence match to sequences of genes YGLO39w and YGL157w, and using activity-based probes that react with YGLO39w and/or Ygl157w. In preferred embodiments using activity-based probes, the probe reacts on a tyrosine in YGLO39w and/or Ygl157w peptides. The various methods can also be used in any combination.

The invention also provides a method for growing an organism under elevated salt conditions in another aspect. The method involves culturing an organism that has at least one gene functionally homologous to genes YGLO39w and/or YGL157w inactivated, under high salt growth conditions, where the high salt growth conditions inhibit growth of an organism that is isogeneic except for having active forms of the inactivated genes.

The high salt conditions can be at various levels or concentrations, e.g., depending on the type of organism. In preferred embodiments, the concentration of sodium ion (e.g., from sodium chloride) is at least 0.3M, 0.4M, 0.5M, 0.7M, 0.8M at least 0.9 M±0.1 M sodium, or is at least 0.9 M sodium. In embodiments involving plants or plant cells, the sodium ion concentration dissolved in the water or medium is preferably at least 0.02M, 0.04M, 0.05M, 0.08M, 0.1M, 0.2M, 0.3M, 0.4M, 0.5M, or even higher.

The organism can be of any type that has the indicated genes, e.g., a yeast, fungus, or plant, or other organism indicated as suitable for this invention. Plants can, for example, include forage plants, such as alfalfa and grasses, grain plants, such as wheat, barley, oat, rice, and corn, and vegetables, as well as many other types of plants

In another aspect, a method for soil improvement is provided. The method involves growing at least one plant having at least one inactivated gene functionally homologous to YGLO39w and/or YGL157w in soil containing a high level of salt, and removing the plants, or at least portions of the plants, following a growth period of at least 10 days. Removed plant portions contain elevated levels of salt. Thus, removal of the plants or plant parts removes excess salt. In certain embodiments, the concentration of dissolved sodium resulting from the combination of sodium in the soil and in any water provided is as shown in the preceding aspect.

In many cases, the plants will be grown for longer times, e.g., at least 30 days, 60 days, 90 days, 180 days, 1 year, 2 years, 5 years, 10 years, or even longer. In certain embodiments, the plants are annuals or perennials. The growing and removing can be done repetitively, either with replanting and/or with plants that re-grow after cutting. In particular embodiments, the repetition is done at least 2, 5, 10, 20, 40, or more times.

In reference to cells, the term “isolated” means that a cell has been removed from an earlier environment, e.g., a natural environment, or from a mixed population.

The term “significant” is used to indicate that the level of increase is useful to the person making such an increase, and is preferably at least 2-fold, more preferably at least 5- to 10-fold or even more.

It is also advantageous for some purposes that a cell be in purified form. The term “purified” in reference to cells does not require absolute purity (such as a homogeneous culture). Instead, it represents an indication that the cell is relatively more pure than a prior environment, e.g., in the natural environment. Compared to the prior level, this level should be at least 2-5 fold greater, e.g., in terms of cells/mL or cell/cm³. Preferably purified cells do not have other cells present at a level detectable by isolation methods appropriate for the type of cells that may be present.

The term “inactivated”, in reference to a gene, means that gene product from a gene that normally encodes a functional product has been modified or treated such that the gene produces no product, completely inactive product, or sufficiently low level of functional product so that the effects described for this invention are obtained, or that the expression or activity of a gene product has been sufficiently inhibited such that the effect described for this invention are obtained (for genes useful in this invention). Preferably the reduction in gene product activity is at least 80%, more preferably at least 90%, still more preferably at least 95%, and most preferably at least 97, 98, 99, or even 100% (no detectable activity). As with YGLO39w and YGL157w, co-inactivation of more than one gene may produce the desired effect. Such inactivation can be accomplished in various ways, e.g., by deletion of all or part of the gene; inserting another sequence or sequences in the gene; by contacting the gene or corresponding mRNA with sense, antisense, ribozyme, or triple-helix-forming sequences, or combinations thereof; by downregulating expression of the gene; by inhibiting processing of transcript; by inhibiting translation of an mRNA; and/or by inhibiting action of a polypeptide or protein. Reference to inactivation of a gene thus can involve inactivation at the DNA, RNA, and/or protein level unless clearly indicated to be more limited.

The terms “YGLO39w”, “YGLO39w gene”, “YGLO39w gene product” “YGLO39w”, and the like refer to the S. cerevisiae coding sequence, gene, and encoded polypeptide represented by SEQ ID NOs. 1 and 2 in Table 1. Yg1039w refers to the encoded polypeptide.

The terms “YGL157w”, “YGL157w gene”, “YGL157w gene product”, Ygl157w”, and the like refer to the S. cerevisiae coding sequence, gene, and encoded polypeptide represented by SEQ ID NOs. 3 and 4 in Table 2. Ygl157w refers to the encoded polypeptide.

In the context of the sequences of this invention, unless otherwise indicated the terms “sequence match” and “matching sequence” and terms of the like import when used without modification indicate that a sequence has at least a minimum level of nucleotide or amino sequence identity (or similarity) to a reference sequence. Matching nucleotide sequences (e.g., gene sequences or coding sequences) have at least 60% or preferably at least 70% sequence identity (as defined by the maximal base match in a computer-generated alignment of two or more nucleic acid sequences) over at least one sequence window of at least 60 nucleotides, or preferably at least 75, 90, 105, 120, 150, or 200 nucleotides, or over an entire ORF or entire gene, more preferably at least 80% or 85%, still more preferably at least 90%, and most preferably at least 95%.

A polypeptide with sequence match (typically a product of a gene with sequence matching) has at least 20% identity over a window of at least 20 contiguous amino acid residues (or larger window as indicated below), preferably at least 25, 30, 35, 40, 45, or 50 amino acids, more preferably over at least 100 amino acids with the sequence of a reference polypeptide. Most preferably the matching sequence has at least 20% identity over the full-length polypeptide encoded by an ORF or entire gene. Preferably the comparison is against YGLO39w and/or Ygl157w polypeptide (or other functionally homologous polypeptide(s)). Highly preferred are amino acid identity levels of 25%, 27%, 30%, 32%, 35%, 37%, or even higher.

The term “similarity”, or the like may also be used to characterize polypeptide sequence matching. As used herein, the term refers to a measure of sequence matching which matches both identical amino acids and conservatively changed amino acids. As is well-known, in connection with amino acid residues, the term “similar” refers to amino acids in which the substituting amino acid has chemico-physical properties which are similar to that of the substituted amino acid. The similar chemico-physical properties include similarities in charge, bulkiness, hydrophobicity, hydrophilicity and the like. When amino acid similarity is used in the homology comparison, the similarity level is at least 45%, more preferably at least 50%, 55%, 60%, 65%, 70%, or even higher.

For nucleotide or amino acid sequence comparisons where a sequence match is defined by a % sequence identity, the percentage may be determined using BLAST programs (with default parameters (Altschul et al., 1997, “Gapped BLAST and PSI-BLAST: a new generation of protein database search programs, Nucleic Acid Res. 25:3389-3402)). Any of a variety of algorithms known in the art which provide comparable results can also be used, with parameters adjusted to provide equivalent results. Performance characteristics for three different algorithms in sequence searching is described in Salamov et al., 1999, “Combining sensitive database searches with multiple intermediates to detect distant homologues.” Protein Eng. 12:95-100. Another exemplary program package is the GCG™ package from the University of Wisconsin.

Alternatively, sequence matches can be defined as the maximum number of identical (or similar) nucleotide or amino acid residues in a comparison sequence compared to a reference sequence for two equal length sequences when the two sequences are aligned to provide maximum match, without the incorporation of gap penalties or other calculations. Thus, when % identity is used as the measure of sequence match, the % identity is calculated as (number of identical residues)÷(total number of residues in each sequence)×(100). For polypeptides, when similarity is used, the % sequence match (or similarity) is calculated as (number of identical residues+number of similar residues)÷(total number of residues in each sequence)×(100). While the alignment can be done manually, highly preferably the alignment is performed using a computer-based alignment program. Match percentage defined using this alignment and calculation is also referred to herein as “equal length match”.

For nucleic acid sequences, sequence matching may also, or in addition, be characterized by the ability of two complementary nucleic acid strands to hybridize to each other under appropriately stringent conditions that allows hybridization at sequence match levels as described above. Thus, matching nucleotide sequences will distinguishably hybridize with a reference sequence with up to three mismatches in ten (i.e., at least 70% base match in two sequences of equal length). Preferably, the allowable mismatch level is up to two mismatches in 10, or up to one mismatch in ten, more preferably up to one mismatch in twenty. (Those ratios can, of course, be applied to larger sequences.) Hybridizations are typically and preferably conducted with probe-length nucleic acid molecules, preferably 20-100 nucleotides in length. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook et al. (1989) Molecular Cloning: A Laboratory Manual, Cold Spring Harbor University Press, Cold Spring, N.Y.; Ausubel, F. M. et al. (1994) Current Protocols in Molecular Biology. John Wiley & Sons, Secaucus, N.J. Matching gene sequences may thus be identified using any nucleic acid sequence of interest.

A typical hybridization, for example, utilizes, besides the labeled probe of interest, a salt solution such as 6×SSC (NaCl and Sodium Citrate base) to stabilize nucleic acid strand interaction, a mild detergent such as 0.5% SDS, together with other typical additives such as Denhardt's solution and salmon sperm DNA. The solution is added to the immobilized sequence to be probed and incubated at suitable temperatures to preferably permit specific binding while minimizing nonspecific binding. The temperature of the incubations and ensuing washes is critical to the success and clarity of the hybridization. Stringent conditions employ relatively higher temperatures, lower salt concentrations, and/or more detergent than do non-stringent conditions. Hybridization temperatures also depend on the length, complementarity level, and nature (i.e., “GC content”) of the sequences to be tested. Typical stringent hybridizations and washes are conducted at temperatures of at least 40° C., while lower stringency hybridizations and washes are typically conducted at 37° C. down to room temperature (˜25° C.). One of ordinary skill in the art is aware that these conditions may vary according to the parameters indicated above, and that certain additives such as formamide and dextran sulphate may also be added to affect the conditions.

By “stringent hybridization conditions” is meant hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5× Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

The terms “tolerant”, “greater tolerance” and terms of like import refer to the ability of a cell or organism to survive and grow better in a given environmental condition better than a reference cell or organism. Typically in the present invention, the reference cell or organism is a wild type cell or organism, or a cell or organism that is isogeneic except for a specified genetic difference.

The term “salt tolerance” includes tolerance to elevated levels of dissolved salts, e.g., NaCl, but can also include tolerance to elevated levels of other dissolved salts, e.g., potassium, calcium, and magnesium salts.

The terms “corresponding cell” and “isogeneic” refer to a cell or organism that is the same as a cell or organism of interest except for some particular, specified genetic difference. For example, a wild type cell is isogeneic to a mutant of that cell except for that mutation(s). Thus, the terms “isogeneic” means that that a cell or organism has the same genetic complement (except for individual variation) as a reference cell or organism. The term is used to indicate that a mutated or derivative cell or organism has the same genetic background as a reference cell, differing only with respect to a specific genetic change.

In connection with genes and gene products, the term “active forms” indicates that the particular gene is present and expressed in an organism, and the gene product has its normal biological activity.

As used herein, the term “cell” or “cells” includes the conventional biological understanding of cells, but also includes biological entities capable of developing into cells and/or complex organisms, e.g., seeds and spores, and reproductive entities, e.g., pollen. Unless indicated to the contrary, the term includes cells in all contexts, for example, single cells, cultures of single cells, and cells in multi-celled organisms.

The term “organism” has its usual biological meaning, referring to a self-replicating, living entity that is in a natural organizational form.

The term “yeast” is used in its usual biological meaning to refer to fungi that are single-celled during at least part of the life cycle. In some cases yeasts form pseudohyphae consisting of linear chains of incompletely separated budded cells.

The term “fungus” is used in it usual biological meaning, and includes both yeasts and molds. Some fungi have multi-nucleate structures.

The term “spores” refers to a natural storage form of an organism, capable to developing into an active organism under appropriate conditions. Examples include fungal spores.

The term “plant” is used in its usual biological meaning.

Similarly, the term “seeds” is used in its usual biological meaning to refer to a structure formed from the ovule of seed plants following fertilization.

In the context of this disclosure, the term “culturing” refers to a process of growing cells or organisms under conditions that allow increase in size and/or number of cells or organisms, or that are intended to test for such increase in size and/or number. For example, culture includes growth of yeast cells in liquid or solid media culture, as well as growth of plants in soil. Thus, culturing is distinguished from mere storage of cells or organisms.

The term “cell culture” is used to refer to culture of cells as distinguished from culturing multicellular organisms, such as plants. That is, the cells are present as generally separated cells without organization into natural complex structures such as tissues. Commonly, cell culture is carried out with liquid media, with the cells either on a surface or surfaces and bathed by the media, or suspended in the media.

In this description, the term “culture density” refers to the density of cells (for a cell culture) or the density of organisms in a culture. Typically, the term is applied to cell cultures, e.g., cells/mL. In this context, the term “high density” or “high cell density” refers to a density of cells (or organisms) that is greater than that of a reference condition, generally the density provided by a cell that is isogenic except for specified genetic changes, e.g., inactivation of genes functionally homologous to YGLO39w and/or YGL157w.

The phrase “high salt conditions” as used herein, refers to the presence of salt, e.g., sodium chloride (or sodium ion) in solution or in position to become solubilized at a concentration higher than normal for a particular cell type or organisms of interest. Thus, for example, for yeasts in liquid culture, the term refers to the salt concentration in the liquid medium, while for plants in soil, the term refers to the salt concentration in the soil and/or in water available to the plant.

As used herein, the term “kit” refers to a combination of two or more items selected to be suitable for a particular use or combination of uses and packaged together. Preferably a kit is prepared to be suitable for commercial sale.

As used in connection with kits, the term “storage device” means a container able to hold and protect the contents from loss under expected condition. Examples include without limitation, bottles, bags, sleeves, envelopes, vials, tubes, and cans.

In the context of this invention, the term “growth conditions” refers to conditions that allow growth, preferably including increase in numbers, of a reference cell or organism.

The term “exponential growth phase” is used in its usual biological sense to refer to the period of growth of cells (e.g., yeasts) in non-replenished medium during which active growth occurs. When number of cells is plotted in a semi-log plot versus time, the exponential growth phase is shown as a generally linear section of the curve, typically between an upward curving initial growth period (generally representing a lag phase and induction of growth) and a later portion of the curve where the slope decreases as growth in the number of cells substantially slows and usually essentially stops (stationary phase).

In keeping with the preceding description the term “stationary growth phase” or “stationary phase” refers to the period in growth of cells in non-replenished medium during which the increase in the number of cells substantially slows and typically stops. Cells can also be maintained in exponential growth phase in continuous culture, e.g., by replenishment of media and removal of cells.

In reference to a particular cellular product, the term “increased yield” means that a culture produces a greater amount of the product than a reference culture, or a greater amount in a specified time period. The increase may, for example, be due to the presence of a greater density (number) of cells in a particular volume of culture. Preferably the greater amount is at least 20%, more preferably at least 50%, still more preferably at least 75%, and most preferably at least 100% more. The increase can be even greater, e.g., 200%, 300%, 400%, 500%, or more.

The term “cell product” refers to a product produced by the biological actions of a cell or organism. Generally a cell product of interest will be a molecule produced by the cell. The product may be a natural product of the cell, or may be produced due to the presence of exogenous genes or gene mutations.

The term “purifying” refers to a process of separating a particular composition (e.g., a molecule or a cell) from at least some of the other compositions with which it is found. While the particular compositions may be purified to homogeneity, the term also includes lesser levels of separation.

In connection with growth of cells or organisms, or activity of a biomolecule, the terms “inhibit”, “inhibit growth”, and like terms refer to a reduction in activity or growth rate resulting from the presence of an agent(s) or environmental condition.

The term “comestible product” refers to a product that is generally regarded as edible or drinkable by humans. Thus, the term includes both food and drink products.

As used herein, the phrase “process utilizing cell culture” refers to a process in which the presence of the cells is intended and provides a significant and desired effect in the process. Examples include the presence of yeasts or other fungi in fermentation processes in producing beer, wine, soy sauce, raised bread, and cheese. Thus, the term does not include the incidental presence of cells in a process, or the unintended presence of cells that produce an undesirable effect.

The term “fermentation” has its usual meaning in referring to a metabolic process (and the associated culture process) that is not principally a respiration process. Thus, fermentation is a generally anaerobic process.

As used herein, the term “liquid culture” refers to a culture of cells or organisms that is carried out with the cells or organisms primarily suspended in a liquid growth medium.

In reference to genetic mutations, the term “deletion” refers to the removal of one or more nucleotides of a prior nucleic acid sequence. Thus, the term can include the removal of most or all of a coding sequence, or an entire gene. Such deletion can also result in the removal of one or more amino acid residues from an encoded polypeptide. As used in this invention, a deletion preferably inactivates the gene in which the deletion occurs.

In reference to genetic mutations, the term “insertion” refers to the addition of one or more nucleotides in a nucleic acid sequence. In a coding sequence, such insertions can result in a chimeric product if inserted in-frame, or can result in frame shifts. As used in the present invention, insertions are usually heterologous sequence insertions, but may also be autologous sequence insertions. Insertions may be performed either with or without accompanying deletions. As used in this invention, insertions preferably inactivate the gene in which the insertion occurs and/or provides a tag for an encoded product.

In reference to nucleic acid and/or amino acid sequences, the term “sequence alignment” has its usual meaning in referring to a process of matching identical or similar residues in two (or more) sequences to identify the maximum number of matching residues. While the process can be done manually, typically such alignment is performed on a computer using widely available software.

The term “low stringency PCR” refers to a polymerase chain reaction (PCR) process in which amplification occurs even when one or more of the primers used have substantial mismatch from the target sequence. Thus, the process allows amplification of homologous sequences using primers based on a reference sequence.

The term “activity-based probe” refers to a molecule that is used to identify proteins (generally enzymes) of a particular class by reacting at the active site of the target molecule, thereby identifying active members of the class. The probe has a direct or indirect tag (e.g., biotin or a fluorescent moiety, or a light scattering moiety). Activity-based probes and their use are described, for example, in PCT Application PCT/US02/03808, entitled “Activity Based Probe Analysis, PCT Application No. PCT/US02/06234, U.S. Provisional Application No. 60/339,424, entitled “Affinity Labeling of ATP Dependent Enzymes”, U.S. Provisional Application No. 60/363,762, entitled “Tethered Activity-Based Probes and Uses Therefore”, PCT Application No. PCT/US00/34187 (WO 01/77684), entitled “Proteomic Analysis”, and PCT Application No. PCT/US00/34167 (WO 01/77668), entitled “Proteomic Analysis”, all of which are incorporated herein by reference.

Additional aspects and embodiments will be apparent from the following Detailed Description and from the claims.

### BRIEF DESCRIPTION OF THE DRAWINGS

FIG. 1 shows images of a set of two culture plates showing the comparative growth of S. cerevisiae strains that are respectively (from left to right) wild type, YGLO39w/YGL157w double knockout, YGLO39w single knockout, and YGL157w single knockout, in rich medium (YPD) and under high NaCl conditions. As shown in the figure, the double knockout strain grows to a higher density in the high salt conditions than the wild type or single knockout strains.

FIG. 2 is a graph showing the growth curves (optical density) in rich medium for S. cerevisiae strains that are respectively wild type, YGLO39w/YGL157w double knockout, YGLO39w single knockout, and YGL157w single knockout. As shown in the graph, the double knockout strain grows to significantly higher density than the wild type and single knockout strains.

### DETAILED DESCRIPTION OF THE PREFERRED EMBODIMENTS

Introduction

The present invention is based on the surprising discovery that cells can be created that are both salt tolerant and will grow to higher densities than parent cells, e.g., wild type cells. For example, inactivation of specific genes results in the organism being salt tolerant, and/or tolerant to the environmental stress produced by high culture density. The ability to tolerate higher culture density demonstrates that the cells are able to continue actively growing beyond the point where feedback from the environment would cause normal cells to stop growing, e.g., starvation or accumulation of metabolic products. Thus, the cells can be tolerant to starvation conditions and/or higher culture density.

This discovery of the creation of salt tolerant cells is in contrast to earlier methods for producing salt tolerance that generally involved the expression or overexpression of genes that provide salt tolerance, e.g., sodium pump genes, or the administration of a compound that results in salt tolerance. Inactivation of endogenous genes thus provides a mechanism for producing salt tolerant cells or organisms without the introduction of foreign DNA, and without the continuous application of chemicals to induce the salt tolerance.

Exemplary genes whose inactivation provides the salt tolerance and/or tolerance to high culture density were identified in Saccharomyces cerevisiae. The two genes were identified as YGLO39w and YGL157w under the standard nomenclature used for identified open reading frames in S. cerevisiae. The sequences of the two genes, along with the encoded polypeptides are provided in Tables 1 and 2 as SEQ ID NOs. 1-4. Also provided are the sequence accession numbers for the sequences. These two genes had a high level of homology, with greater than 70% identity between the encoded polypeptides. Surprisingly, it was found that inactivation of the YGL157w gene resulted in substantially greater tolerance to elevated level of salt in the growth medium as compared to wild type. In a further unexpected discovery, it was found that the double knockout of both YGLO39w and YGL157w had both substantially greater salt tolerance than even the YGL157w single knockout, and grew to higher density in rich media.

Based on the YGLO39w and YGL157w sequences (and/or the corresponding polypeptide sequences), matching sequences in other organisms, particularly other fungi and plants can be identified using conventional sequence matching software. For example, searching currently available sequences from various organisms revealed the following exemplary sequences with significant amino acid sequence matching to the Ygl039w gene product:

| Species | | |
|---|---|---|
| aa's | genbank id | identical aa's/total |
| Oryza sativa (rice) | 4581047 | 82/293 |
| Zea mays (corn) | 14030554 | 88/291 |
| Arabidopsis thaliana | 15219749 | 112/350 |
| Arabidopsis thaliana | 15223845 | 98/301 |
| Arabidopsis thaliana | 15239741 | 101/291 |
| Vigna unguiculata (cowpea) | 7431406 | 112/342 |
| Eucalyptus gunnii (cider tree) | 7431409 | 104/299 |
| Malus x domestica (apple tree) | 7488627 | 101/296 |
| Vigna radiata (mung bean) | 5852203 | 99/294 |
| Populus tremuloides (aspen) | 7239228 | 98/338 |
| Saccharum officinarum | 3341511 | 103/342 |
| Vitis vinifera (wine grape) | 1888485 | 97/333 |

These sequence examples demonstrate that matching sequences to the specifically identified yeast genes are widely distributed in plants, and are not limited to yeast and other fungi. Thus, as is described in more detail below, the sequences identified above as well as other matching sequences that are identified can be tested to characterize functional homologs of the Ygl09w and Ygl157w gene products. Additional functional homologs can be identified and characterized in other organisms using known methods.

The surprising determination that the double gene knockout had salt tolerance and actively grew to higher density than wild type provides a beneficial method for providing modified cells with commercially valuable properties without creating transgenic organisms. The salt tolerance allows a double knockout organism to actively grow in a broader range of conditions than the wild type organism, while the tolerance of high culture density provides more efficient use in fermentations and other culture processes.

Saccharomyces cerevisiae Gene Identification and Knockout Creation

Open reading frames YGLO39w and YGL157w were sequenced and these designations assigned in the sequencing of S. cerevisiae. However, the functions of these genes were not known. Indeed, the discovery that inactivation of these genes resulted in salt tolerance and tolerance to high culture density was entirely fortuitous. These genes were initially tagged in a process using fluorophosphonate activity-based probes to identify active serine hydrolases in Saccharomyces cerevisiae. Two of the proteins identified had sequences that did not appear consistent with serine hydrolase function.

Based on the polypeptide sequences, the corresponding nucleic acid sequences were identified as YGLO39w and YGL157w. As the genome of S. cerevisiae has been sequenced, this identification was done by computer searching. To characterize these gene products, the genes were each cloned and overexpressed in bacteria. It was confirmed that both of the gene products were labeled by the fluorophosphonate probes. The specific labeled amino acids from each protein were identified using conventional mass spectrometry methods. Each protein was labeled on a tyrosine residue (in the motif fYny(c/s): the capitalized Y is the tyrosine that gets modified). It had been previously reported that neither of these genes is essential to viability of yeast (all of the genes have been individually deleted in a massive effort, but little detailed characterization had been done on most of the resulting viable knockouts in the Stanford yeast deletion project (see the Web site with the address name following www being: sequence.stanford.edu/group/yeast_deletion_project/deletions3.html)).

To make the double knockout, standard PCR-based methods were used to knock out the YGL157w gene in a purchased strain that had YGLO39w deleted. The double knockout was not make by simple mating as the two genes are close to one another on the same chromosome, resulting in low probability of recombining. The purchased strain was generated as part of the public deletion effort (cited above) and purchased from Research Genetics, a unit of Invitrogen. Most of the YGL157w gene was deleted as described in Example 2.

Characterization of Double Knockout Mutant

The double knock-out yeast that resulted were viable and had normal-appearing colony morphology and microscopic morphology. In looking for phenotypes of the double knockout, the yeast were grown under various conditions, looking for conditions where the double knockout was less robust than wild-type yeast. However, the opposite was found; the double knockout was able to grow to higher density than the wild type yeast. Additionally, the salt resistance of the double knockout strain was greater than that of the wild type strain, and greater than that of either of the single knockout strains.

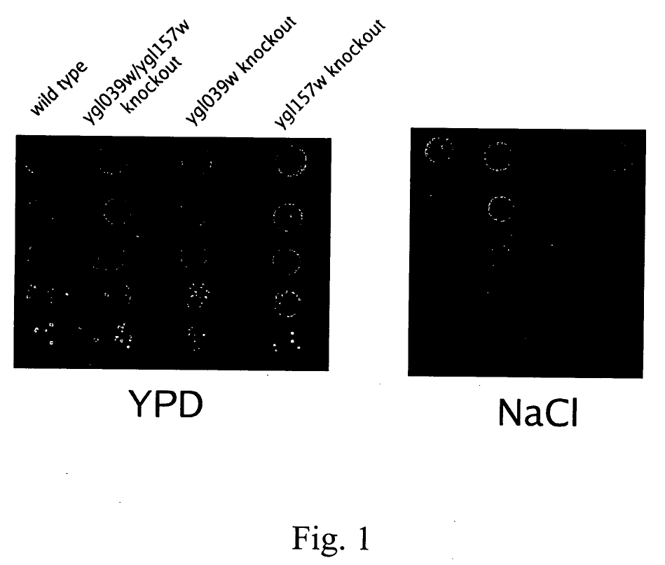

In fact, the double knockout provided a synergistic contribution to salt tolerance. As shown in FIG. 1, the YGLO39w single knockout strain was only slightly (if at all) more tolerant to 0.9 M NaCl than the wild type strain, while the YGL157w single knockout grew to approximately 10× the number of cells in rich medium with 0.9 M NaCl as the wild type. In contrast, the double knockout grew to approximately 100× the number of cells as wild type.

As indicated, the double knockout strain also grew to high densities during active growth. In effect, the double knockout strain continued in exponential growth phase to substantially higher cell density than either the wild type or the single gene knockouts.

Identification of Sequence Matching Genes

As indicated above, genes having sequence match to YGLO39w and YGL157w can be located in a variety of different organisms and inactivated and tested for use in the present invention. Sequence matching genes can be identified using various known techniques. In locating sequence matching genes, the simplest method is to use sequence comparisons of known sequences. Preferably, the sequence comparison is carried out at the amino acid level, but can also be done using the corresponding nucleotide sequences. While such comparisons can be done manually, highly preferably computer-based analysis is used. In this analysis, two sequences are aligned to maximize the matching amino acids (or nucleotides). Usually the matches are for identity, but for amino acids, similarity can be used.

For identification of sequence matches in the present invention, an amino acid sequence identified as a sequence match has at least 20% identity over a window of at least 50 amino acids, preferably over at least 100 amino acids, and most preferably over the full-length polypeptide encoded by an ORF compared against Ygl039w and/or Ygl157w (or other functionally homologous polypeptide). Highly preferred are amino acid identity levels of 25%, 27%, 30%, 32%, 35%, 37%, or even higher. If amino acid similarity is used, the similarity level is at least 45%, more preferably at least 50%, 55%, 60%, 65%, 70%, or even higher.

Once polypeptides with sequence matching are identified in an organism, those polypeptides can be tested, as described below, to determine which constitute functional homologs, i.e., which sequence matches produce the effects demonstrated for Ygl009w and Ygl157w inactivation.

In addition to use on known sequences, sequence alignment and sequence match determinations will generally be performed on sequence matches (or other potential functional homologs) revealed by other techniques. For example, sequence matches can be identified using low stringency PCR and sequencing of the amplified sequences. Likewise, potential functional homologs can be identified using activity-based probes, followed by characterization of the protein (e.g., by sequencing). The corresponding coding sequences can then be identified, e.g., by searching for a nucleic acid sequence encoding the particular polypeptide or portion thereof, or by using degenerate probes and/or low stringency PCT to obtain corresponding coding sequences. Typically hybridization probe screening is performed using cDNA libraries, while PCR may be performed using genomic and/or cDNA libraries. The various techniques can also be used in combination.

As indicated, low stringency PCR can be used to identify matching sequences, a well-known approach. In general this method involves the use of PCR primers that are designed based on a known sequence, in this case the nucleotide sequences of YGLO39w and YGL157w genes (or identified functional homologs). Hybridization conditions (as part of the amplification conditions) are used that allow a selected level of mismatches of the primers. Thus, if a sequence is present in the sample to which the primers can hybridize under the selected conditions, extension can occur, resulting in amplification of the target sequence despite the possible presence of partially mismatched primers. Various sets of primers with differing sequences can be tested to provide primer set(s) that provide appropriate amplification. Primers can be selected corresponding to unique or low redundancy regions. Additionally, once sequence matches are identified in particular cells or organisms, the sequences of those sequence matches can be used in further primer (or probe) design.

In an alternative approach, instead of (or in addition to) proceeding initially with low stringency PCR, the presence of sequence matches in an organism can be demonstrated using hybridization of sequence matches to a reference sequence (e.g., YGLO39w and/or YGL157w gene sequences) (i.e., hybridization probes). Preferably coding sequences are used. Detection of hybridized sequences can be performed in various ways. When the hybridization is carried out using non-amplified nucleic acid from an organism, the detection method should be highly sensitive, e.g., capable of detecting one or a few copies. An example of such a detection method is the use of light scattering particles (e.g., gold and/or silver). See, e.g., Yguerabide et al. U.S. Pat. No. 6,214,560 Alternatively, hybridization can be used in conjunction with low stringency PCR. In this case the detection method does not need to be as sensitive. In addition to light scattering, labels such as fluorescent and radio-isotope labels can be used. As with the use of low stringency PCR, probes can be selected corresponding to unique or low redundancy target regions.

Once the presence of matching sequences is confirmed, the full coding sequence can be isolated and sequenced using conventional methods. Sufficient material for sequencing can be isolated using hybridization (effectively used as affinity chromatography), but preferably is provided by amplification. Such amplification can be performed as low stringency PCR. Primers can be based on the hybridization probes. Preferably the initial sequence match determination provides full-length coding sequence, or even full-length gene. If full-length sequence is not provided initially, full-length sequence can be obtained using the partial sequence already provided to provide exact match probes to isolate larger fragments or cDNAs, providing the missing sequence portions.

For example, the partial sequence can be used to provide probes suitable for high stringency hybridization (preferably providing perfect match). Such probes can be used to detect genomic or cDNA clone inserts to identify a clone that includes the full coding sequence or full gene. The clone preferably is constructed with adjacent sequences that allow convenient isolation and sequencing of the insert.

Once the full sequence is identified, or even when only a portion of the coding sequence is known, the gene product can be inactivated in the process of identifying functional homologs, as described below.

Possible functional homologs can also be identified, at least initially, using activity-based probes. As described Ygl039w and Ygl157w were initially identified as being of interest using fluorophosphonate activity-based probes that usually react with the active site serine hydrolases. In this case, the probe reacted with anomalously nucleophilic tyrosines in these two gene products. Thus, such probes, or other activity-based probes that react with the Ygl039w or Ygl157w gene products (or identified functional homolog) can be used to react with possible functional homologs. Such possible functional homologs can be at least partially sequenced as was done for Ygl039w and Ygl157w, and the residue with which the probe reacted identified using conventional mass spectrometry techniques.

Based on the polypeptide sequencing, the coding sequence can be identified. The coding sequence identification can be done immediately by computer sequence search if the coding sequence is available on computer. If not available on computer, the coding sequence can be obtained using conventional methods involving hybridization and/or PCR using degenerate probes and/or primers to locate and/or amplify the homologous nucleotide sequence. The nucleotide sequence can then be sequenced by conventional methods. The polypeptide sequence encoded by the nucleotide sequence can then be checked to confirm that the correct nucleotide sequence has been obtained.

Inactivation of Sequence Match Genes

As indicated, once sufficient portions (or preferably all) of the nucleic acid sequences of sequence matches in an organism are available (e.g., from sequence databases and/or from separate determination), those sequence matches can be inactivated to test for the creation of salt tolerance and/or tolerance to high culture density. S. cerevisiae was found to contain four sequence matches, but only Ygl039w and Ygl157w provided the salt tolerance and culture density tolerance when inactivated. Thus, when more than two sequence match genes are identified in a cell or organism, they should be inactivated in a manner allowing discrimination of the functional homologs that provide the functional effects of salt tolerance and/or culture density tolerance when inactivated, preferably when inactivated in combination.

This discrimination can be done in a systematic manner, e.g., by inactivating the sequence matches in all combinations. However, other schemes can be followed. For example, in a first test, all identified matching sequence genes can be inactivated in a single strain, thereby establishing whether the necessary functional homologs have been included in the set of sequence matches (so long as inactivation of the full set of sequence match genes does not result in the cell or organism being non-viable or having a phenotype that interferes with the salt tolerance and culture density determinations. Alternatively, in a first test, overlapping subsets of the sequence match genes can be inactivated, so that the identity of functional homologs can be further narrowed.

Gene inactivation can be carried out in a number of different ways. An exemplary method is the use of homologous recombination to create inactivating deletions in a target gene, often with accompanying insertion of heterologous sequence. This approach was used for the inactivation of YGL157w.

Another example of the method is described in Glazer et al., U.S. Pat. No. 5,776,744, entitled METHODS AND COMPOSITIONS FOR EFFECTING HOMOLOGOUS RECOMBINATION. Further examples are provided in Wang et al., 1995, “Targeted mutagenesis in mammalian cells mediated by intracellular triple helix formation”, Mol. Cell. Biol. 15(3):1759-1768; Rooney et al., 1995, Anti-parallel, intramolecular triplex DNA stimulates homologous recombination in cells”, Proc. Natl. Acad. Sci. 92:2141-2144; Kim et al., 1994, “Parallel DNA triplexes and homologous recombination”, in Structural Biology: The State of the Art, Proceeding of the Eighth Conversation, Adenine Press, Albany, N.Y., pp. 67-74.

A system that is often used for plants utilizes the Cre-lox system. A description of the use of this system is provided in Bayley et al., “Exchange of Gene Activity in Transgenic Plants Catalyzed by the Cre-Lox Site-Specific Recombination System”, Plant Molecular Biology 18:353-361 (1992). Further examples are provided in Qin, et al., Proc. Natl. Acad. Sci. USA 91:1706-1710 (1994); Sauer, Methods in Enzymology 225:890-900 (1993).

Another exemplary method that can be used is based on inactivating insertions, e.g., transposon insertions or insertions by Agrobacterium tumefaciens T-DNA (or similar element). (Bouchez and Höfte, 1998, Plant Physiol. 118:725-732; Martienssen, 1998, Proc. Natl. Acad. Sci. USA 95:2021-2026; Azpiroz-Leehan and Feldmann, 1997, Trends Genet. 13:152-156.) An example of a systematic insertional knockout approach is provided by the Arabidopsis Knockout Facility creation of knockouts of Arabidopsis thaliana. (See, e.g., Sussman et al., 2000, Plant Physiol. 124:1465-1467.) Additional guidance on gene silencing is provided in Vaucheret and Fagard, 2001, Trends Genet. 17(1):29-35.

In addition to methods that involve inactivating gene mutations, for purposes of testing matching gene products to discriminate the functional homologs, methods can be used that involve expression blocking at the mRNA level or at the transcription level. One such method uses RNA interference (RNAi). (See, e.g., Chuang and Meyerowitz, 2000, Proc. Natl. Acad. Sci. USA 97:4985-4990; Schmitz et al., 2002, Proc. Natl. Acad. Sci. 99:1064-1069.) In general, this method involves the introduction of dsRNA-expressing constructs targeted to a particular gene into a cell. The introduced constructs can be designed to be present in extrachromasomal vectors, or to integrate into the host cell genome.

Another such method uses ribozymes (or other catalytic nucleotide-containing molecule, such as catalytic DNA (all referred to herein as ribozymes)) to cleave particular mRNAs in a site specific manner, thereby inhibiting expression of the particular message. Typically ribozymes are either expressed in the cell, e.g. from a vector, or stablilized ribozymes are introduced exogenously, e.g., using liposome delivery. Such ribozymes and methods for utilizing them are known in the art.

Another method for inhibiting expression at the mRNA level involves the use of antisense (or sense) molecules. As with ribozymes, antisense molecules are nucleotide-containing molecules that are either expressed in a cell, or are introduced into a cell exogenously. For introduction into a cell, the antisense molecules (or ribozymes) are highly preferably stablilized by the incorporation of nucleotide analogs and/or non-ribonucleotide moieties. Such antisense molecules generally act by blocking translation and/or by inducing cleavage of the message by endogenous cell components. An example of this approach is described in Baucher et al., Plant Mol. Biol. 39:437-447 (1999). Additional inhibitions are described in Lapierre et al., Plant Physiol. 119:153-164 (1999) and MacKay et al., Science 277:235-239 (1997).

In many cases, the ribozyme or antisense methods will not completely eliminate expression, but will reduce the level of expression sufficiently to provide an indicator of whether a sequence match is a functional homolog.

When using ribozymes or antisense molecules, testing can proceed in various ways. For example, in order to confirm the presence of functional homologs, ribozymes or antisense molecules can be targeted to sequences that are identical between some or all of the sequence matches. Once the presence of functional homologs is confirmed, different combination can be tested to select the functional homologs, with the ribozymes or antisense molecules targeted to sequences that differ sufficiently between sequence matches to provide either specific, or specific combination, inhibition. Similarly, initial targeting can be to sequences that differ sufficiently to provide first stage testing of combinations of sequence matches. Also, for both ribozymes and antisense molecules, multiple ribozymes and/or antisense molecules can be targeted to a particular transcript, thereby providing a higher level of inhibition.

Expression inhibition can also be performed by inhibiting transcription by forming triple helix structures. Methods and constructs for performing such inhibition are known in the art. (See, e.g., Blume et al., 1992, Nucl. Acids Res. 20; 1777; Grigoriev et al., 1993, Proc. Natl. Acad. Sci. USA 90:3501; 1 ng et al., 1993, Nucl. Acids Res. 21:2789.)

Additional examples of gene suppression methods are provided in WO 01/49844; WO 00/68374; WO 00/49035; WO 00/44914; WO 99/61631; WO 99/53050; WO 99/49029; WO 9932619; WO 92/15680; WO 98/36083; WO 98/53083; U.S. Pat. No. 5,107,065; U.S. Pat. No. 5,759,829; U.S. Pat. No. 5,190,931; U.S. Pat. No. 5,208,149; U.S. Pat. No. 5,272,065; U.S. Pat. No. 5,5,922,602; U.S. Pat. No. 5,231,020; U.S. Pat. No. 5,283,184; and U.S. Pat. No. 6,232,122.

Methods such as those mentioned above or other techniques for inhibiting or blocking expression of a gene can be used in the present invention. Combinations of two or more of the different techniques can also be used for a particular gene or genes in a particular organism. Such combinations can be particularly useful if difficulty is encountered in inactivating or sufficiently inhibiting particular genes. Whatever the method or combination of methods used to inactivate sequence match genes, the inactivation and testing of sequence match genes provides selection of the sequence matches that are also functional homologs. With that selection, cell or organism strains are provided (or can be provided by mutational inactivation) and can be used in the various processes for which the original strain was appropriate, as well as in new processes made practical by the increased salt tolerance and/or increased cell density tolerance.

While yeasts and other fungi generally can typically be cultured in liquid media or on solid media, for inactivation, testing, and/or culture in connection with complex organisms, it is often desirable to utilize cell or tissue culture. Appropriate techniques for providing cell and tissue cultures from plants are well-known. Exemplary descriptions are provided in Maki, et al., “Procedures for Introducing Foreign DNA into Plants” in Methods in Plant Molecular Biology & Biotechnology, Glich, et al., (Eds. pp. 67-88 CRC Press, 1993); and by Phillips, et al., “Cell-Tissue Culture and In-Vitro Manipulation” in Corn & Corn Improvement, 3rd Edition; Sprague, et al., (Eds. pp. 345-387) American Society of Agronomy Inc., 1988.

Introduction of Nucleic Acid into Cells

In order to accomplish nucleic acid-based gene inactivations (or for other applications such as introduction of a heterologous gene, coding sequence, or regulatory sequence), it is often necessary to introduce nucleic acid sequences into the respective cells. A number of such methods are known and can be utilized, with the specific selection depending on the particular type of cells. Yeast cells can, for example, be transformed by converting yeast cells into protoplasts, e.g., using zymolyase, lyticase, or glusulase, followed by addition of the nucleic acid and polyethylene glycol (PEG). The PEG-treated protoplasts are then regenerated by culturing in a growth medium, e.g., under selective conditions. See, e.g., Beggs, Nature 275:104-108 (1978); and Hinnen et al., Proc. Natl. Acad. Sci. USA 75:1929-1933 (1978). Another method does not involve removal of the cell wall, instead utilizing treatment with lithium chloride or acetate and PEG and then grown on selective media. See, e.g., Ito et al., J. Bact. 153:163-168 (1983). A variety of methods for yeast transformation, integration of genes into the yeast genome, and growth and selection of yeast strains are described in Current Protocols in Molecular Biology, Vols. 1 and 2, Ausubel et al., eds., John Wiley & Sons, New York (1997).

An exemplary method for the transformation of S. cerevisiae is as follows. Yeast strains are cultured overnight in YPD (yeast extract, peptone, dextrose) medium at about 30° C. The resulting culture is diluted to an A600 of about 0.2 in about 200 ml YPD medium and incubated at about 30° C. until the A.sub.600 reaches approximately 0.8. The cells are pelleted by centrifugation and are washed in about 20 ml sterile water. The pelleted yeast cells are then resuspended in about 10 ml TEL (10 mM Tris pH7.5, 1 mM EDTA, 0.1 M LiAcetate pH 7.5) buffer. The cells are pelleted by centrifugation and again resuspended in about 2 ml TEL. About 100 microgram of well-sheared single stranded DNA and plasmid DNA are added to an eppendorf tube. To this tube is added about 100 microliter of competent yeast cells, followed by mixing. To the cell/DNA mixture is added about 0.8 ml of 40% PEG-3350 in TEL, followed by thorough mixing. This mixture is incubated for about 30 minutes at 30° C., followed by a heat shock for 20 minutes at 42° C. The mixture is centrifuged to remove the supernatant and pellet the cells. The yeast cell pellet is washed with about 1 ml TE, pelleted again by centrifugation, and then plated on selective media.

Techniques applicable to other fungi are also well-known and can be utilized. See, e.g., the Beggs and Hinnen references cited above, as well as Yelton et al., Proc. Natl. Acad. Sci. USA 81:1740-1747 (1984); Russell, Nature 301:167-169 (1983); and U.S. Pat. No. 4,935,349.

Likewise, techniques for transforming a variety of different plant species are well-known. See, for example, Weising et al., Ann. Rev. Genet. 22:421-477 (1988). Exemplary methods utilize electroporation (e.g., Fromm et al., Proc. Natl. Acad. Sci. USA 82:5824 (1985); Shimamoto et al., Nature 338:274-276 (1989)), PEG poration (e.g., Paszkowski et al, EMBO J. 3:2717-2722 (1984)), particle bombardment (e.g., Klein et al., Nature 327:70-73 (1987); Li et al, Plant Cell rep. 12:250-255)); microinjection of plant cell protoplasts or embryogenic callus, and transformation vectors, e.g., Agrobacterium mediated transformation (for example, Agrobacterium tumefaciens-mediated transformation) (preferably with T-DNA flanking regions) (e.g., Horsch et al., Science 233:496-498 (1984); Fraley et al., Proc. Natl. Acad. Sci. USA 80:4803 (1983); Hiei et al., Plant J 6:271-282 (1994); Rogers et al., Methods in Enzymology 153:253-305 (1987)). General descriptions of plant expression vectors and reporter genes and transformation protocols can be found in Gruber, et al., “Vectors for Plant Transformation, in Methods in Plant Molecular Biology & Biotechnology” in Glich, et al., (Eds. pp. 89-119, CRC Press, 1993).

Additional examples of transformation techniques (and generation of transgenic plants) are provided in Sautter et al., U.S. Pat. No. 5,877,023; Sandford, Trends in Biotechnology 6:299-302; Christou et al., Proc. Natl. Acad. Sci. USA 86:7500-7504 (1989); Christou et al., Plant Physiol. 87:671-674 (1988); Oard et al., Plant Physiol. 92:334-339 (1990); Tomes et al., U.S. Pat. No. 6,258,999; D'Halliun, U.S. Pat. No. 6,140,553; Bytebier et al, Proc. Natl. Acad. Sci. USA 84:5345-5349 (1987); U.S. Pat. No. 5,164,310; U.S. Pat. No. 5,187,073; U.S. Pat. No. 5,177,010; WO 92/09696; EP 0604662 A1; and EP 0672752 A1.

In the case of plant cells and tissues, following transformation, it can be useful to regenerate whole plants. Such regeneration can be carried out by known methods suitable for the particular plant type. In many cases regeneration is performed using embryos, protoplasts, meristematic cells, callus, pollen, leaves, anthers, roots, root tips, flowers, seeds, pods or stems. An example of a regeneration method is described in Tomes et al., U.S. Pat. No. 6,258,999, which is indicated as appropriate for a number of different plants, including species from the genera Fragaria, Lotus, Medicago, Onogrychis, Trifolium, Trigonella, Vigna, Citrus, Linum, Geranium, Manicot, Daucus, Arabidopsis, Brassica, Raphanus, Sinapis, Atropa, Capsicum, Datura, Hyoscyamus, Lycopersionn, Nicotiana, Solanum, Petunia, Digitalis, Marorana, Cichorium, Helianthus, Lactuca, Bromus, Asparagus, Antirrhinum, Hemerocallis, Nemesia, Pelargonium, Panicum, Pennisetum, Ranunculus, Senecio, Salpiglossis, Cucumis, Browallia, Glycine, Lolium, Zea, Triticum, and Sorghun, thus, including major cereal crops such as maize, rye, barley, wheat, sorghum, oats, millet, rice, sunflower, rape seed, and soybean, as well as the livestock feed or forage crop alfalfa. Plant regeneration is also described in D'Halliun, U.S. Pat. No. 6,140,553. General methods of culturing plant tissues are provided for example by Maki, et al., “Procedures for Introducing Foreign DNA into Plants” in Methods in Plant Molecular Biology & Biotechnology, Glich, et al., (Eds. pp. 67-88 CRC Press, 1993); and by Phillips, et al., “Cell-Tissue Culture and In-Vitro Manipulation” in Corn & Corn Improvement, 3rd Edition; Sprague, et al., (Eds. pp. 345-387) American Society of Agronomy Inc., 1988.

It can also be useful to have control of expression of introduced genes. Examples of such control is provided in Oliver, U.S. Pat. No. 5,925,808 and references cited therein.

Single Gene Inactivation

In addition, it is useful to test the single gene inactivations (single gene knockouts). As was shown for the YGL157w single knockout (see FIG. 1), a single knockout strain can provide a useful level of salt tolerance compared to wild type. For example, the YGL157w single knockout strain provided approximately 10× the number of cells as either the wild type strain or the YGLO39w single knockout in 0.9M NaCl. While that level of tolerance was significantly lower than the salt tolerance of the double knockout, the single knockout result indicates that single knockouts of functional homologs in other organisms is also useful for creating salt tolerant cells and organisms.

For identification of useful single knockout homologs, gene inactivation methods can be used either directly with single gene knockouts of the individual sequence match genes, preferably of all sequence match genes in a particular cell or organism, or by focusing from strains bearing multiple knockouts shown to include one or more functional homologs.

Testing Cells with Inactivated Sequence Match Genes

Testing of cells with inactivated sequence match genes can be done using conventional growth assays. Thus, cells are grown in an appropriate growth media for the cell type, but with the salt concentration (or other dissolved component or components) at higher than normal concentration. The ability of the strains with inactivated sequence matches to grow in the stress condition is determined and compared to the ability of a reference strain to grow under those conditions.

Testing for increased culture density can be performed generally as described herein for the exemplary S. cerevisiae strains. For example, in separate cultures, the number of the respective strains are normalized and the respective cultures are grown under the same conditions for a period sufficient for at least the wild type, parent, or other reference strain to go through active growth and into stationary phase (or significantly reduced growth rate). Preferably the growth is continued for sufficient time that all strains pass to stationary phase. At least the maximum cell densities are determined (e.g., as optical density or viable cell numbers or densities or concentrations). Preferably the cell densities are determined at multiple time points, thereby allowing construction of growth curves. The terminal densities (or total numbers) and/or growth curves provide comparative growth indicators.

In conducting growth tests, culture conditions (including growth media) should be selected as appropriate for the particular cells or organisms. For yeast, exemplary media includes YPD (1% yeast extract, 2% peptone, 2% dextrose, water (weight % s)).

Use of Cells and Organisms with Inactivated Functionally Homologous Genes

Either single, double, or other knockout combinations can be used in various ways, depending on the type of organism or cell. Generally, the knockout strains are useful in any process in which it would be useful to have cells or organisms grow in a higher than normal concentration of salt (or other dissolved components), or actively grow to higher concentration than the wild type or other reference strain), or both. In addition, tolerance of such stress conditions can open additional new applications for particular types of organisms. In many cases, the strains or cell types of the present invention can reduce costs and/or increase output of a desired product.

For example, for cultures (cell or organisms) that are grown in liquid culture, tolerance to high culture density allows the culture to be effectively run with a denser culture. This applies to both batch culture and continuous culture applications. For batch culture, the tolerant organisms allow the culture to be run to higher cell density, generally providing a greater amount of product for that batch and/or faster output. For continuous culture, the tolerant strain culture can be run at higher culture density while still maintaining active cell growth. Those higher call densities provide greater cell product output from the culture.

The cells and organisms can be used in many different production processes, including production of comestible products. For established processes, the strains routinely used can be modified by inactivating the respective functionally homologous genes in that strain, thereby providing a strain more tolerant to culture stresses. In addition, the modified strains can be cultured in the process at higher density while maintaining active growth.

Examples of such established processes include those used in production of beer, wine, fermented soy sauce, and cheese. Those processes are well-known in the respective fields.

The use of the modified organisms is also very useful in cell culture production of products that are isolated from the culture. Some products are isolated from the medium, while others are isolated from the organisms after the cells are lysed or broken. The present invention is applicable in both cases.

In addition, for applications involving plants, the present strains allow growth in conditions with high levels of salt in the soil and/or water. This properly provides at least two types of applications. In the first, the plants will still grow when water containing elevated levels of salt (e.g., NaCl) and/or other dissolved minerals and other ions is supplied, thus allowing use of slightly brackish water. In the second, plants can be grown in soil that contains a high level of salt or other components that can mobilize in the water, allowing growth in marginal soil. Further, if a plant is selected that both grows in the high salt conditions and has a high salt concentration in at least some tissues, the plants can be grown and harvested. Harvesting removes the salt concentration in the plant, thus removing salt from the soil. Repetition of the process can remove substantial amounts of salt from the soil. This process can be carried out over a number of years. In such use the plants both stabilize the soil and improve the soil by the salt removal. For the salt removal applications, preferably the plant is a perennial that provides a plurality of harvestings each year. The crop can itself be a commercial crop, etc., alfalfa, wheat, corn, barley, and rice, among others. This process is also applicable to removal of other soil components that can mobilize in water and be taken up by the plants.

### EXAMPLES

### Example 1

### Labeling and Identification of Ygl039w and Ygl157w

A culture of yeast strain BY4743 (ATCC strain 201390) was grown in rich media to an OD600 of approximately 5. Cells were sedimented and resuspended in phosphate buffered saline, pH 7.4. Following reduction of disulfide bonds in the cell wall with DTT, cells were lysed with a high-pressure homogenizer. Cell lysates were spun at 15,000×g, and the supernatent from the 15,000×g spin was spun at 100,000×g. The fraction from which Ygl039w and Ygl157w were purified was the supernatant from the 100,000×g centrifugation. 10 mg of lysate was reacted with 4 microM of FP-tetramethylrhodamine (an activity-based probe for serine hydrolases) for 1 h at room temperature.

Following the reaction, proteins were denatured with urea, disulfide bonds were reduced with DTT and cysteines alkylated with iodoacetamide. Following gel filtration into ammonium acetate/urea buffer, labeled proteins were isolated by chromatography. Protein samples that have been labeled with an activity-based probe were passed through an anti-rhodamine antibodies affinity purification column (Sepaharose), where the bound antibodies recognize the rhodamine probes attached to the labeled proteins. Labeled proteins that are purified in this manner were separated by standard 1-dimensional SDS-PAGE (12.5%) and visualized using laser-induced fluorescence from a flat-bed laser scanner. Fluorescent proteins are physically removed from the gel using a spot picker (Amersham).

Gel spots containing fluorescent proteins were washed and treated with trypsin. Tryptic peptides from the labeled proteins were then separated by liquid chromatography in-line to an ion trap mass spectrometer. The peptides were typically analyzed using 2 dimensions of mass spectrometry. In the first dimension, the mass to charge ratio of the whole peptide (or peptide-probe conjugate) is observed. In the second dimension, the peptide is fragmented in predictable fashion, commonly at the amide bonds. This fragmentation results in species being generated that have, for example, 1, 2 and 3 amino acids removed from a terminus of the peptide. The mass spectrometer provides the means to observe the masses of the intact, -1, -2 and -3 fragments. Since the masses of all the amino acids is known, this method enables peptide sequencing in the mass spectrometer. Data generated from mass spectrometer was compared to databases of protein sequences generated from S. cerevisiae, from which the sample was derived, to identify proteins that contained the predicted peptide sequence. Two proteins identified from a 37 kD region in this manner were Ygl039w and Ygl157w.

The amino acid sequences of Ygl039w and Ygl157w were used to obtain the corresponding nucleic acid sequences from the known S. cerevisiae ORF sequences.

Similarly, identification and/or sequencing can be performed for other proteins, e.g., from other organisms. For organisms in which the polypeptide and/or ORF sequences have not previously been determined, the amino acid sequence (preferably the entire polypeptide) can be used to obtain the corresponding nucleic acid sequence by conventional methods. For example, corresponding coding sequences or genes can be obtained using degenerate hybridization probe sets and/or using low stringency PCR (which can use degenerate primer design), with the probe and/or primers preferably based on the amino acid sequence. Preferably the probes and/or primers are designed to be complementary to unique or low degeneracy regions.

### Example 2

### Inactivating Ygl039w and Ygl157w

A PCR product that contained the URA2 sequence (amplified from pYES2 from Invitrogen) flanked by sequence from the beginning and the end of YGL157w was made. Transformation of the PCR product into the YGLO39w knockout strain led to recombination of the flanking sequences, resulting in the replacement of almost the entire YGL157w gene with that of URA2—allowing for the selection of recombinants and the deletion of YGL157w. Proper integration was verified by PCR. The oligonucleotides used for the deletion PCR were:

| | |
|---|---|
| ATGACTACTGATACCACTGTTTTCGTTTCTGGCGCA | (SEQ ID NO. 5) |
| ACCGGTTTCATTGCTCTACACATTCCtttttcaatg | |
| ggtaataa | |
| TTAGGCTTCATTTTGAACTTCTAACATTTGCGCCGC | (SEQ ID NO. 6), |
| GGGTGTCAACTATGCAATCCTTTAAtagcttttcaa | |
| ttcaattca, | |

The 5′ primer is listed first. For each, the capital letters show sequence match to parts of YGL157w, while the lower case sequence anneals to sequences around the URA2 gene.

### Example 3

### Characterization of Cells with Inactivated Ygl039w and Ygl157w

Two assays were performed to look for growth phenotypes of the double deletion strain relative to the two single deletion strains and wild-type yeast. In the first assay, the four strains were grown in YPD overnight and diluted to an OD600 of 5.0. One to ten serial dilutions of the OD600 5.0 stock were made down to 1/10,000. Three microliters of each dilution were spotted onto a YPD plate, and a YPD plate supplemented with 0.9M NaCl. The plates were incubated at 30° C. for 2-4 d. The results are shown in FIG. 1, demonstrating that the double deletion strain grew significantly better in the 0.9 M NaCl supplemented medium compared to the wild type and to the single deletion strains. Also apparent is that the YGL157w single deletion strain grew significantly better than either the wild type or the YGLO39w single deletion strain.

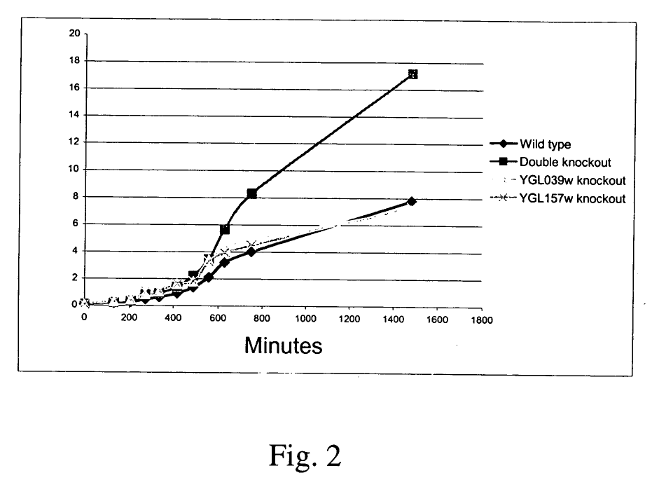

In a second experiment, the four strains were grown overnight in YPD, diluted to normalize their OD600 and grown in YPD at 30 deg C. Over a period of approximately one day, samples of each culture were removed at several time points and the OD600 of the culture was taken. The results were plotted and are shown in FIG. 2. As shown in the figure, the double deletion strain grew to significantly greater density than any of the other 3 strains. All the other strains grew to quite similar densities.

### Example 4

### Identification of Sequence Matches of Ygl039w and Ygl157w in Other Species

A basic protein-protein BLAST search was conducted using Ygl039w a query sequence. Sequence similar proteins were found in many species of plants, including rice, corn and Arabidopsis. Exemplary sequence matches are identified above. These proteins are typically on the order of 25% identical over 250-300 amino acids. As such, the proteins are considered to be highly sequence similar and most likely are functionally similar.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, using other cell types and organisms are all within the scope of the present invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

Thus, additional embodiments are within the scope of the invention and within the following claims.

TABLE 1

YGLO39w
>gi|1322522|emb|CAA96741.1|ORF YGLO39w [Saccharomyces
cerevisiae]

| | |
|---|---|
| YgIO39w polypeptide sequence | (SEQ ID NO. 1) |

MTTEKTVVFVSGATGFIALHVVDDLLKTGYKVIGSGRSQEKNDGLLKKFKSNPNLSMEIV
EDIAAPNAFDKVFQKHGKEIKVVLHIASPVHFNTTDFEKDLLIPAVNGTKSILEAIKNYA
ADTVEKVVITSSVAALASPGDMKDTSFVVNEESWNKDTWESCQANAVSAYCGSKKFAEKT
AWDFLEENQSSIKFTLSTINPGFVFGPQLFADSLRNGINSSSAIIANLVSYKLGDNFYNY
SGPFIDVRDVSKAHLLAFEKPECAGQRLFLCEDMFCSQEALDOLNEEFPQLKGKIATGEP
GSGSTFLTKNCCKGDNRKTKNLLGFQFNKFRDCIVDTASQLLEVQSKS

| | |
|---|---|
| YGLO39w nucleotide sequence | (SEQ ID NO. 2) |

| | |
|---|---|
| | ATGACTACTG AAAAAACCGT TGTTTTTGTT TCTGGTGCTA CTGGTTTCAT |
| | TGCTCTAGAC GTAGTGGACG ATTTATTAAA AAGTGGTTAC AAGGTCATCG |
| 101 | GTTCGGGTAG GTCCCAAGAA AAGAATGATG GATTGCTGAA AAAATTTAAG |
| 151 | AGCAATCCCA ACCTTTCAAT GGAGATTGTC GAAGACATTG CTGCTCCAAA |
| 201 | CGCTTTTGAC AAAGTTTTTC AAAAGCACGG CAAAGAGATC AAGGTTGTCT |
| 251 | TGCACATAGC TTCTCCGGTT CACTTCAACA CCACTGATTT CGAAAAGGAT |
| 301 | CTGCTAATTC CTGCTGTGAA TGGTACCAAG TCCATTCTAG AAGCAATCAA |
| 351 | AAATTATGCC GCAGACACAG TCGAAAAAGT CGTTATTACT TCTTCTGTTG |
| 401 | CTGCCCTTGC ATCTCCCGGA GATATGAAGG ACACTAGTTT CGTTGTCAAT |
| 451 | GAGGAAAGTT GGAACAAAGA TACTTGGGAA AGTTGTCAAG CTAACGCGGT |
| 501 | TTCCGCATAC TGTGGTTGCA AGAAATTTGC TGAAAAAACT GCTTGGGATT |
| 551 | TTCTCGAGGA AAACCAATCA AGCATCAAAT TTACGCTATC AACCATCAAC |
| 601 | CCAGGATTTG TTTTTGGCCC TCAGCTATTT GCCGACTCTC TTAGAAATGG |
| 651 | AATAAATAGC TCTTCAGCCA TTATTGCCAA TTTGGTTAGT TATAAATTAG |
| 701 | GCGACAATTT TTATAATTAC AGTGGTCCTT TTATTGACGT TCGCGATGTT |
| 751 | TCAAAAGCTC ATTTACTTGC ATTTGAGAAA CCCGAATGCG CTGGCCAAAG |
| 801 | ACTATTCTTA TGTGAAGATA TGTTTTGCTC TCAAGAAGCG CTGGATATCT |
| 851 | TGAATGAGGA ATTTCCACAG TTAAAAGGCA AGATAGCAAC TGGCGAACCT |
| 901 | GGTAGCGGCT CAACCTTTTT GACAAAAAAC TGCTGCAAGT GCGACAACCG |
| 951 | CAAAACCAAA AATTTATTAG GATTCCAATT TAATAAGTTC AGAGATTGCA |
| 1001 | TTGTCGATAC TGCCTCGCAA TTACTAGAAG TTCAAAGTAA AAGCTAA |

TABLE 2

YGL157w
>gi|1322748|emb|CAA96869.1|ORF YGL157w [Saccharomyces
cerevisiae]

| | |
|---|---|
| Ygl157w polypeptide sequence | (SEQ ID NO. 3) |

MTTDTTVFVSGATGFIALHIMNDLLKAGYTVIGSGRSQEKNDGLLKKFNNNPKLSMEIVE
DIAAPNAFDEVFKKHGKEIKIVLHTASPFHFETTNFEKDLLTPAVNGTKSILEAIKKYAA
DTVEKVIVTSSTAALVTPTDMNKGDLVITEESWNKDTWDSCQANAVAAYCGSKKFAEKTA
WEFLKENKSSVKFTLSTINPGFVFGPQMFADSLKHGINTSSGIVSELIHSKVGGEFYNYC
GPFIDVRDVSKAHLVAIEKPECTGQRLVLSEGLFCCQEIVDILNEEFPQLKGKIATGEPA
TGPSFLEKNSCKFDNSKTKKLLGFQFYNLKDCIVDTAAQMLEVQNEA

| | |
|---|---|
| YGL 157w nucleotide sequence | (SEQ ID NO. 4) |

| | |
|---|---|
| | ATGACTACTG ATACCACTGT TTTGGTTTCT GGCGCAACCG GTTTCATTGC |
| | TCTACACATT ATGAACGATC TGTTGAAAGC TGGCTATACA GTGATCGGCT |
| 101 | CAGGTAGATC TCAAGAAAAA AATGATGGCT TGCTCAAAAA ATTTAATAAC |
| 151 | AATCCCAAAC TATCGATGGA AATTGTGGAA GATATTGCTG CTCCAAACGC |
| 201 | CTTTGATGAA GTTTTCAAAA AACATGGTAA GGAAATTAAG ATTGTGCTAC |
| 251 | ACACTGCCTC CCCATTCCAT TTTGAAACTA CCAATTTTGA AAAGGATTTA |
| 301 | CTAACCCCTG CAGTGAACGG TACAAAATCT ATCTTGGAAG CGATTAAAAA |
| 351 | ATATGCTGCA GACACTGTTG AAAAAGTTAT TGTTACTTCG TCTACTGCTG |
| 401 | CTCTGGTGAC ACCTACAGAC ATGAACAAAG GAGATTTGGT GATCACGGAG |
| 451 | GAGAGTTGGA ATAAGGATAC ATGGGACAGT TGTCAAGCCA ACGCCGTTGC |
| 501 | CGCATATTGT GGCTCGAAAA AGTTTGCTGA AAAAACTGCT TGGGAATTTC |
| 551 | TTAAAGAAAA CAAGTCTAGT GTCAAATTCA CACTATCCAC TATCAATCCG |
| 601 | GGATTCGTTT TTGGTCCTCA AATGTTTGCA GATTCGCTAA AACATGGCAT |
| 651 | AAATACCTCC TCAGGGATCG TATCTGAGTT AATTCATTCC AAGGTAGGTG |
| 701 | GAGAATTTTA TAATTACTGT GGCCCATTTA TTGACGTGCG TGACGTTTCT |
| 751 | AAAGCCCACC TAGTTGCAAT TGAAAAACCA GAATGTACCG GCCAAAGATT |
| 801 | AGTATTGAGT GAAGGTTTAT TCTGCTGTCA AGAAATCGTT GACATCTTGA |
| 851 | ACGAGGAATT CCCTCAATTA AAGGGCAAGA TAGCTACAGG TGAACCTGCG |
| 901 | ACCGGTCCAA GCTTTTTAGA AAAAAACTCT TGCAAGTTTG ACAATTCTAA |
| 951 | GACAAAAAAA CTACTGGGAT TCCAGTTTTA CAATTTAAAG GATTGCATAG |
| 1001 | TTGACACCGC GGCGCAAATG TTAGAAGTTC AAAATGAAGC CTAA |

1. A cell comprising:
at least one inactivated gene functionally homologous to YGLO39w or YGL157w or both; wherein said cell has greater tolerance to high sodium chloride concentration than a corresponding cell comprising active forms of said genes.

2. The cell of claim 1, wherein said cell is a yeast cell.

3. The cell of claim 2, wherein said cell is a Saccharomyces cerevisiae cell.

4. The cell of claim 1, wherein said cell is an Aspergillus or Saccharomyces cell.

5. The cell of claim 1, wherein said cell is a fungal cell.

6. The cell of claim 1, wherein said cell is a plant cell.

7. The cell of claim 1, wherein said cell culture is a high density cell culture.

8. The cell of claim 1, wherein said cell is in high salt conditions.

9. The cell of claim 1, wherein said cell further comprises at least one additional genetic characteristic providing increased salt tolerance.

10. The cell of claim 9, wherein said additional genetic characteristic provides increased sodium pump activity.

11. The cell of claim 1, wherein said cell is also tolerant to increased culture density.

12. The cell of claim 11, wherein said increased culture density is at least 100% increased.

13. A population of salt tolerant cells in a high salt environment, wherein said cells comprise at least one inactivated gene functionally homologous to Ygl039w or Ygl157w or both, wherein said cells are rendered salt tolerant; and
said cells grow in said high salt environment.

14-24. (canceled)

25. A salt tolerant organism, comprising cells in which at least one gene functionally homologous to Ygl039w or Ygl157w or both is inactivated.

26. The salt tolerant organism of claim 25, wherein said organism is a fungus.

27. The salt tolerant organism of claim 25, wherein said organism is a plant.

28. The salt tolerant organism of claim 25, wherein said inactivation of genes said confers salt tolerance.

29. The salt tolerant organism of claim 25, wherein said inactivation of said genes confers an ability to grow to high density.

30. The salt tolerant organism of claim 25, wherein said inactivation of said genes confers tolerance to solutions with elevated dissolved components.

31. A yeast comprising at least one artificial genetic alteration, wherein said yeast is more salt tolerant and actively grows to higher culture density than an isogeneic yeast without said at least one artificial genetic alteration.

32. The yeast of claim 31, wherein said at least one artificial genetic alteration comprises inactivation of least one gene.

33. A yeast strain that is more salt tolerant and grows to a higher density in rich media than the corresponding wild type strain.

34. The yeast strain of claim 33, wherein said corresponding wild type strain is a designated standard wild type strain deposited in an approved depository.

35. A Saccharomyces cerevisiae yeast strain that grows to an OD⁶⁰⁰ of at least 15 in YPD medium.

36. A Saccharomyces cerevisiae yeast strain that grows to a higher density in YPD with 0.9M NaCl than strain BY4743.

37. A mutated cell that is more tolerant to the presence of salt and grows to higher density under growth conditions than the parent cell.

38. The cell of claim 37, wherein said mutation provides inactivation of at least one gene.

39. A culture kit comprising
a plurality of cells comprising inactivated genes functionally homologous to YGLO39w or YGL157w or both packaged in a storage device.

40. The kit of claim 39, further comprising instructions for growing said cells.

41. The kit of claim 39, further comprising a culture medium suitable for growing said cells.

42-44. (canceled)

45. A method for growing cells to high density, comprising:
culturing cells comprising inactivated genes functionally homologous to genes YGLO39w or YGL157w or both under growth conditions until high cell density is obtained during exponential growth phase, wherein said high cell density is greater than that obtained in exponential growth phase under the same growth conditions for cells that are isogeneic except for having active genes functionally homologous to genes YGLO39w and YGL157w.

46-50. (canceled)

51. (canceled)

52. A method for providing increased yield of a cell product, comprising:
culturing cells comprising inactivated genes functionally homologous to genes YGLO39w or YGL157w or both under growth conditions; and
purifying said cell product.

53. The method of claim 52, wherein said cells are grown in exponential growth phase to a cell density that is at least 50% greater than that obtained in exponential growth phase under the same growth conditions for cells that are isogeneic except for having active genes functionally homologous to genes YGLO39w and YGL157w.

54. The method of claim 52, wherein said cells are grown in exponential growth phase to a cell density that is at least 100% greater than that obtained in exponential growth phase under the same growth conditions for cells that are isogeneic except for having active genes functionally homologous to genes YGLO39w and YGL157w.

55-58. (canceled)

59. The method of claim 52, wherein said cells are grown to stationary phase and the density of said cells at stationary phase is greater than that obtained at stationary phase under the same growth conditions for cells that are isogeneic except for having active genes functionally homologous to genes YGLO39w and YGL157w.

60. A method for growing cells in high salt conditions, comprising:
culturing cells comprising inactivated genes functionally homologous to genes YGLO39w or YGL157w or both under high salt growth conditions, wherein said high salt growth conditions inhibit growth of cells that are isogeneic except for having active genes functionally homologous to genes YGLO39w and YGL157w.

61. The method of claim 60, wherein said high salt conditions comprises at least 0.9 M sodium.

62-65. (canceled)

66. A method for producing a comestible product in a process utilizing cell culture, comprising:
culturing cells comprising inactivated genes functionally homologous to YGLO39w or YGL157w or both in said process.

67. The method of claim 66, wherein said product is selected from the group consisting of soy sauce, beer, wine, cheese, and yeast-based nutritional supplements.

68. The method of claim 66, wherein said process is a fermentation.

69. The method of claim 66, wherein said culturing is in liquid culture.

70. The method of claim 69, wherein said liquid culture is grown to stationary phase, and the density of said cell at stationary phase is greater than that obtained under the same growth conditions by cells that are isogeneic except for having active genes functionally homologous to genes YGLO39w and YGL157w.

71. The method of claim 66, wherein said liquid culture is maintained as an actively growing continuous culture, and the cell density of said continuous culture is greater than that at which cells that are isogeneic except for having active genes functionally homologous to genes YGLO39w and YGL157w maintain actively growing culture under the same culture conditions.

72-75. (canceled)

76. A method for producing a cell tolerant to high levels of dissolved materials, comprising
inactivating forms of genes having sequence match to YGLO39w or YGL157w or both in said cell; and
testing said cells for tolerance to high levels of dissolved material.

77-79. (canceled)

80. The method of claim 76, wherein said inactivating involves deletion of at least a part of said genes.

81. The method of claim 76, wherein said inactivating involves insertions in said genes.

82. The method of claim 76, wherein cells comprising genes with sequence match to YGLO39w and YGL157w comprise at least 3 different genes with sequence match to YGLO39w and YGL157w, and cells with all combinations of genes with sequence match to YGLO39w and YGL157w are inactivated and tested.

83. The method of claim 76, wherein said high levels of dissolved materials comprises high levels of sodium chloride.

84. The method of claim 76, wherein said high levels of dissolved materials comprise high levels of cell culture products.

85-89. (canceled)

90. A method for producing a cell that grows to high density, comprising inactivating genes with sequence match to YGLO39w or YGL157w or both in cells; and testing said cells for the ability to maintain exponential growth at densities higher than that provided by cells that are isogeneic except for having active forms of said genes.

91-93. (canceled)

94. The method of claim 90, wherein said inactivating involves deletion of at least a part of at least one of said genes.

95. The method of claim 90, wherein said inactivating involves insertions in at least one of said genes.

96. The method of claim 90, wherein cells with inactivated genes with sequence match to YGLO39w and YGL157w contain at least 3 different genes with sequence match to YGLO39w or YGL157w or both, cells with all combinations of genes with sequence match to YGLO39w and YGL157w are inactivated and tested.

97-101. (canceled)

102. A method for growing an organism under elevated salt conditions, comprising:
culturing an organism comprising at least one inactivated gene functionally homologous to genes YGLO39w or YGL157w or both under high salt growth conditions, wherein said high salt growth conditions inhibit growth of an organism that is isogeneic except for having active genes functionally homologous to genes YGLO39w and YGL157w.

103. The method of claim 102, wherein said high salt conditions comprises at least 0.9 M sodium.

104-108. (canceled)

109. A method for soil improvement, comprising:
growing at least one plant comprising inactivated at least one gene functionally to YGLO39w or YGL157w or both in soil containing a high level of salt; and
removing at least portions of said plants following a growth period of at least 10 days, wherein said portions contain elevated levels of salt.

110. The method of claim 109, wherein said plants are grown for a period of at least 90 days.

111. The method of claim 110, wherein said growing and said removing are performed a plurality of times.

112. The method of claim 45, wherein said cells are grown to stationary phase.

113. The method of claim 112, wherein said cells are grown to stationary phase and the density of said cells at stationary phase is greater than that obtained at stationary phase under the same growth conditions for cells that are isogeneic except for having active genes functionally homologous to genes YGLO39w and YGL157w.
